(19) United States
(12) Patent Application Publication
(10) Pub. No.: US 2013/0338543 A1
(43) Pub. Date: Dec. 19, 2013

(54) PATIENT DETERIORATION DETECTION

(71) Applicants: Guenter Gegner, Tuebingen (DE); Sebastian Hebler, Herrenberg (DE)

(72) Inventors: Guenter Gegner, Tuebingen (DE); Sebastian Hebler, Herrenberg (DE)

(73) Assignee: KONINKLIJKE PHILIPS N.V., EINDHOVEN (NL)

(21) Appl. No.: 14/001,679

(22) PCT Filed: Feb. 22, 2012
(86) PCT No.: PCT/IB2012/050796

§ 371 Date: Aug. 27, 2013

Related U.S. Application Data
(60) Provisional application No. 61/447,836, filed on Mar. 1, 2011.

(51) Int. Cl. A61B 5/00; A61B 5/11

(52) U.S. Cl. CPC A61B 5/7275; A61B 5/0002; A61B 5/11; A61B 5/746; A61B 5/7207
USPC 600/595; 600/300

(57) ABSTRACT

A system (108) detects deterioration of a patient of a medical institution. A processor(152)receives attended physiological data for the patient from a user interface or a patient monitoring system (102) and obtains a baseline patient score from the attended physiological data and a scoring table. The scoring table is received from a memory (118). The processor (152) further receives physiological data, including at least one of unattended physiological data and attended physiological data, which includes measurements of one or more of the physiological parameters of the patient, from a patient monitoring system (102) for the patient. Scores of the physiological data measurements are compared to corresponding most recent attended physiological data measurements using the scoring table. A clinician is notified of patient deterioration if the score of one or more physiological data measurements has deteriorated as compared to the scores of the corresponding most recent attended physiological data measurements.

The present application relates generally to patient monitoring. It finds particular application in conjunction with detecting patient deterioration, and will be described with particular reference thereto. However, it is to be understood that it also finds application in other usage scenarios, and is not necessarily limited to the aforementioned application.

Vital signs acquisition for patients is typically performed at periodic intervals, such as every few hours, by clinicians. The frequency depends on the severity of a patient and the resources of the treating medical institution. As healthcare costs and global competition have increased over the years, medical institutions have been forced to implement cost saving measures. These cost saving measures include caring for more patients than have been cared for in the past, reducing staff, replacing staff with less educated and/or less qualified personnel, transferring patients from the intensive care unit (ICU) to the general ward earlier than has been done in the past, and so on. The net effect is that medical institutions are unable to physically gather vital signs from patients as frequently as they once were and are becoming increasingly dependent upon patient monitoring systems to acquire vital signs. Patient monitoring systems are typically worn by patients and/or placed at patient bedsides and acquire common physiological data, such as pulse oxygen saturation, temperature, electrocardiography (ECG), and the like.

One problem with placing reliance upon patient monitoring systems to acquire vital signs is that the vital signs may be unreliable. Measurements may be skewed and/or distorted by movement artifacts and/or from not knowing conditions when the measurements were taken. For example, vital signs may be skewed and/or distorted depending upon whether a patient is resting or walking Another problem with placing reliance upon patient monitoring systems is that current systems do not evaluate vital sign measurements collected during the interim between attended vital signs acquisition for patient deterioration. Attended vital signs are vital signs obtained with the supervision of a clinician, whereas unattended vital signs are vital signs obtained without the supervision of a clinician. As a result, patient deterioration may not be recognized early enough to intervene in a timely manner. Yet another problem with placing reliance upon patient monitoring systems is that they may become disconnected from other supports systems of a typical medical institution, such that the ability to alert caretakers of deterioration is diminished and/or disabled.

The present application provides a new and improved systems and methods for detecting patient deterioration which overcome the above-referenced problems and others.

In accordance with one aspect, a deterioration detection system for detecting deterioration of a patient of a medical institution is provided. The system includes one or more processors programmed to receive attended physiological data for a patient. The attended physiological data includes automatically or manually collected measurements of physiological parameters of the patient and, in certain embodiments, manual assessments of physiological parameters. The processors are further programmed to obtain a patient score for the patient from the attended physiological data and a scoring table and receive physiological data, including at least one of unattended physiological data and attended physiological data, for the patient. The physiological data includes measurements of one or more of the physiological parameters of the patient. The processors are further programmed to compare the measurements of the physiological data to corresponding measurements in most recent attended physiological data using the scoring table to determine any change in the patient score. Even more, the processors are programmed to notify a clinician of patient deterioration in response to a physiological parameter of the physiological data deteriorating as compared to a corresponding physiological parameter in the most recent attended physiological data.

In accordance with another aspect, a method for detecting deterioration of a patient of a medical institution is provided. Attended physiological data for the patient is received. The attended physiological data includes automatically or manually collected measurements of physiological parameters of the patient and, in certain embodiments, manual assessments of physiological parameters. A patient score for the patient is obtained from the attended physiological data and a scoring table, and physiological data, including at least one of unattended physiological data and attended physiological data, for the patient is received. The physiological data includes measurements of one or more of the physiological parameters of the patient. In other words, the physiological data typically includes measurements for a subset of the parameters of most recent attended physiological data. The measurements of the physiological data are compared to corresponding measurements in the most recent attended physiological data using the scoring table to determine any change in the patient score. A clinician of patient deterioration is notified in response to a physiological parameter of the physiological data deteriorating as compared to a corresponding physiological parameter in the most recent attended physiological data.

In accordance with another aspect, a method for verifying deteriorated unattended physiological data for a patient is provided. The deteriorated unattended physiological data, including measurements of one or more physiological parameters of the patient, is received. In response to intermittently receiving unattended physiological data, a patient monitoring system is controlled to take additional measurements of at least one of the physiological parameters a predetermined number of times in one or more predetermined intervals. Further, supplemental unattended physiological data is received for the patient, including the additional measurements. In response to continuously receiving unattended physiological data, measurements of at least one of the physiological parameters are captured a predetermined number of times in one or more predetermined intervals. The measurements of the deteriorated unattended physiological data are compared to corresponding measurements of the supplemental unattended physiological data or the captured measurements.

One advantage is that patient deterioration can be detected in real time.

Another advantage is that patient deterioration can be detected from trend data.

Another advantage is that patient deterioration detection is event based.

Another advantage is that medical institutions can reduce the frequency with which caretakers manually acquire vital signs from patients.

Another advantage is that workflows of medical institutions are improved.

Another advantage is that patient safety is improved.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

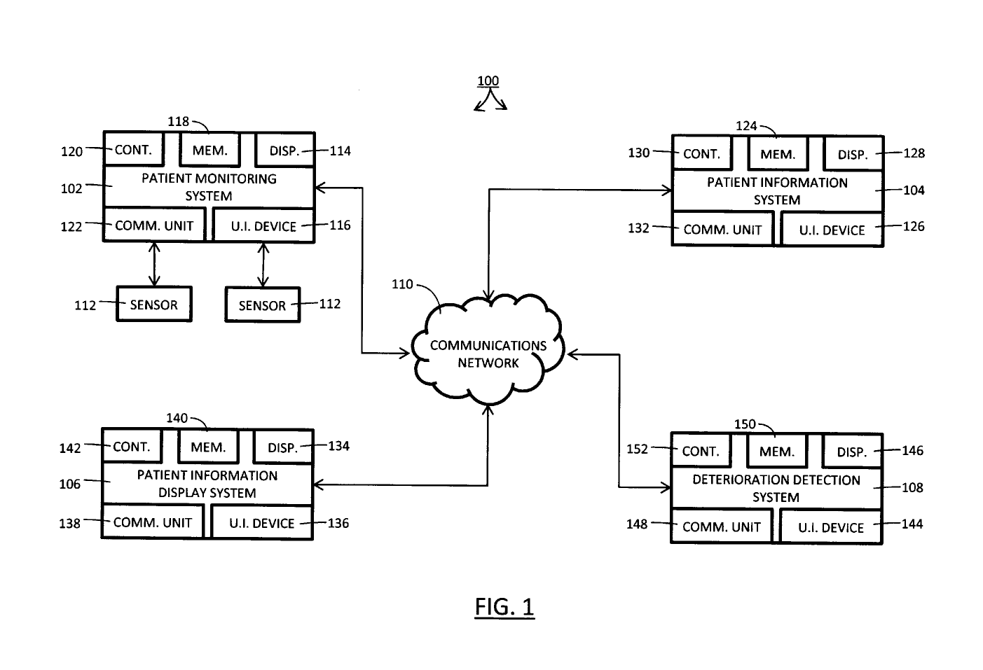

FIG. 1 illustrates an information technology (IT) infrastructure of a medical institution according to aspects of the present disclosure.

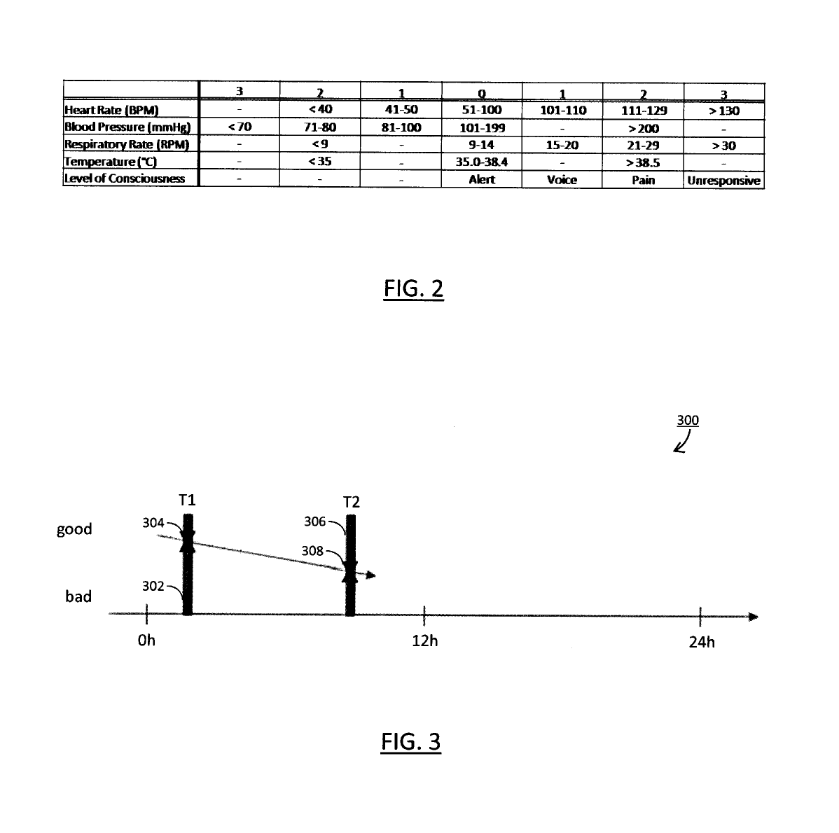

FIG. 2 is one embodiment of a scoring table generated for a deterioration detection system of the present disclosure.

FIG. 3 is an example timeline illustrating a patient's condition and the receipt of baseline data by a deterioration detection system of the present disclosure.

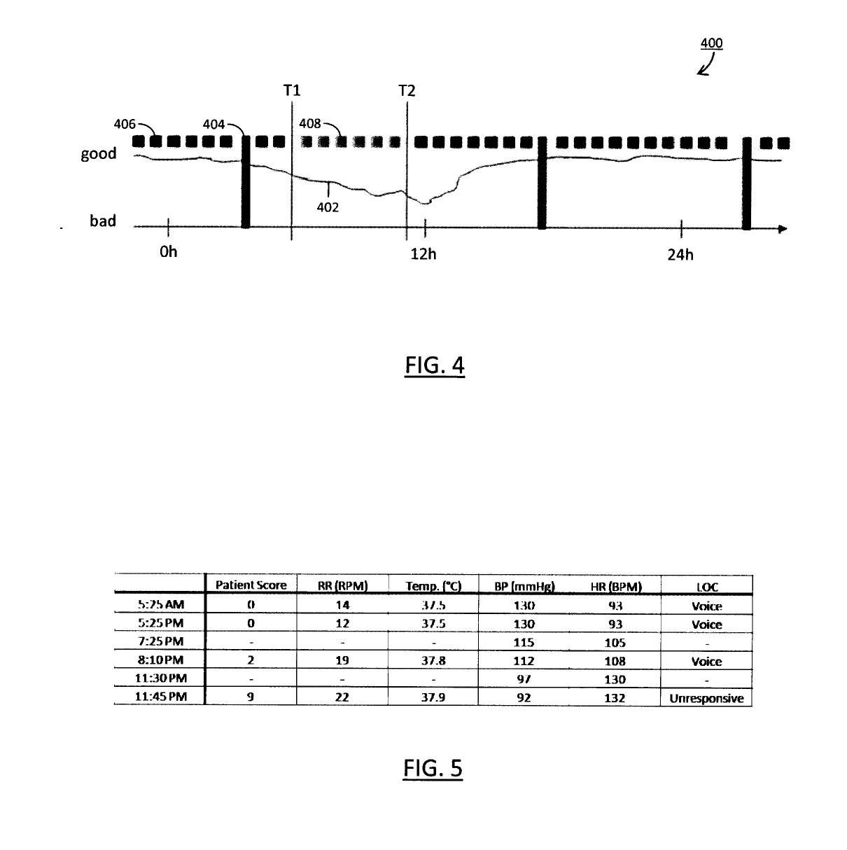

FIG. 4 is an example timeline illustrating a patient's condition and the receipt of baseline data and unattended physiological data by a deterioration detection system of the present disclosure.

FIG. 5 is one example table of events illustrating baseline data and unattended physiological data encountered by a deterioration detection system of the present disclosure.

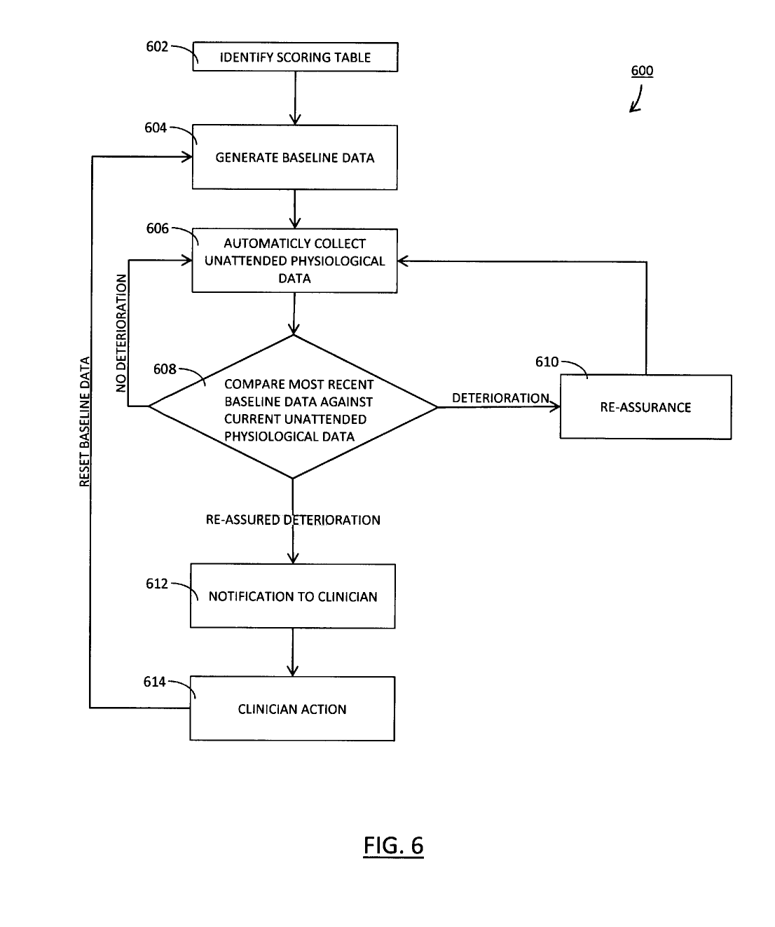

FIG. 6 is a flow chart of a method for detecting deterioration of a patient according to aspects of the present disclosure.

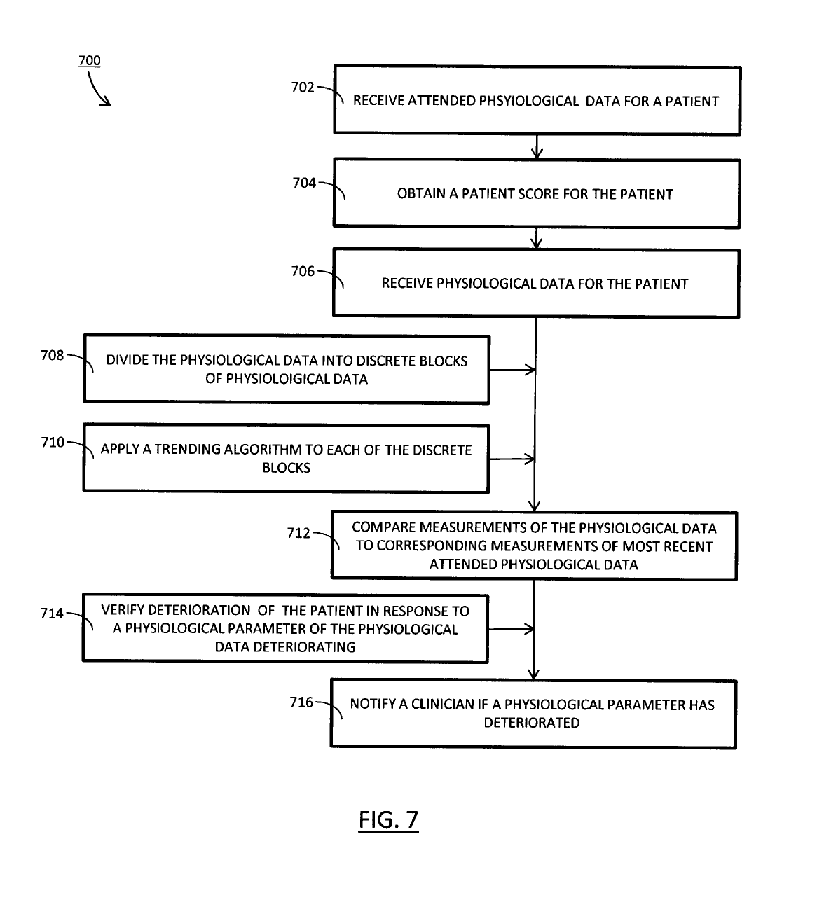

FIG. 7 is a block diagram of a method for detecting deterioration of a patient according to aspects of the present disclosure.

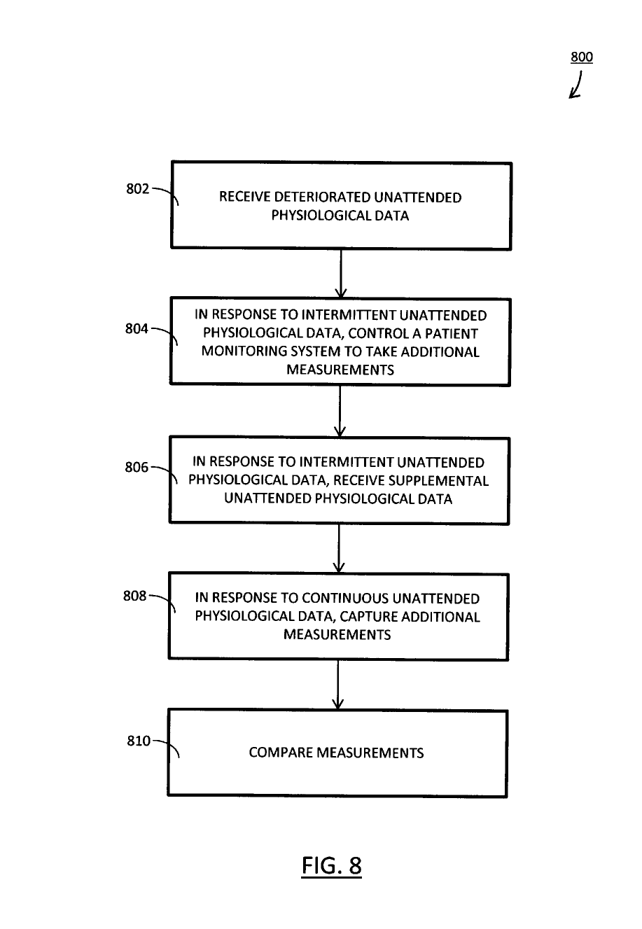

FIG. 8 is a block diagram of a method for verifying unattended physiological data according to aspects of the present disclosure.

With reference to FIG. 1, a block diagram illustrates one embodiment of an information technology (IT) infrastructure 100 of a medical institution, such as a hospital. The IT infrastructure 100 suitably includes one or more patient monitoring systems 102, a patient information system 104, one or more patient information display systems 106, a deterioration detection system 108, and the like, interconnected via a communications network 110. It is contemplated that the communications network 110 includes one or more of the Internet, a local area network, a wide area network, a wireless network, a wired network, a cellular network, a data bus, and the like.

The patient monitoring systems 102 obtain unattended physiological data for patients (not shown) cared for by the medical institution. Unattended physiological data is obtained automatically without the supervision of a clinician and is indicative of measurements of physiological parameters (or vital signs) of the patients, such as heart rate, temperature, blood oxygen saturation, and the like. It is any timely ordered random sequence of measurements. Typically, each of the patient monitoring systems 102 is associated with, and obtains unattended physiological data for, a single patient, but patient monitoring systems associated with multiple patients are contemplated. The unattended physiological data is typically obtained continuously or intermittently. When the unattended physiological data is obtained intermittently, events trigger the acquisition of the unattended physiological data. Events include, for example, timer events (for periodic acquisition), manually triggered events, asynchronous events, network events, and so on. A network event is an event from a component of the IT infrastructure 100, such as the deterioration detection system 108, which triggers the acquisition of physiological data from the patient monitoring systems 102. When the unattended physiological data is obtained continuously or frequently, a trending algorithm (e.g., average, median, peak-finding, etc.) is applied, in one embodiment, to break the stream of physiological data into discrete blocks of unattended physiological data. For example, a continuous stream of unattended physiological data can be separated into blocks of a predetermined duration and a trending algorithm can be applied to each block. Further, when a network event is received, the patient monitoring systems 102 can decide if requested measurements are already available or if a new measurement need to be acquired.

One or more sensors 112 suitably obtain the unattended physiological data. However, it is also contemplated that the physiological data is obtained from other components of the IT infrastructure 100, such as lab equipment, components with user input devices, and so on. The sensors 112 measure physiological parameters of the patients and generate physiological data indicative thereof. In certain embodiments, the sensors 112 include one or more electrocardiographic (ECG) electrodes, blood pressure sensors, SpO2 sensors, pulse sensors, thermometers, respiratory sensors, exhaled gas sensors, noninvasive blood pressure (NBP) sensors, and the like. Typically, the sensors 112 are disposed on the person of a patient and external to the patient monitoring systems 102. However, sensors local to the patient monitoring systems are contemplated. Where the sensors 112 are external, the physiological data can be obtained via a databus, such as a serial bus, a universal serial bus (USB), or the like; a body coupled network; a Bluetooth, a ZigBee, a wired or a wireless network; a medical body area network (MBAN); or the like.

One challenge with unattended physiological data is that it may be unreliable. Measurements can become skewed and/or distorted by movement artifacts and/or from not knowing under what condition the measurements were obtained. To address this problem, in certain embodiments, the patient monitoring systems 102 take one or more actions to ensure unattended physiological data accurately reflects the physiological state of the patients. The actions can include requesting manual confirmation of measurements from clinicians via one or more displays 114 and/or one or more user input devices 116 of the patient monitoring systems 102. Additionally or alternatively, the actions can include obtaining additional data, such as data pertaining to motion, patient activity, body posture, and so on, to allow determination of the state of the patients for which unattended physiological data relates. For example, data indicating whether a patient is in motion (e.g., climbing stairs) can be obtained from an accelerometer incorporated in one or more of the sensors 112. When the additional data indicates the measurements of the unattended physiological data are likely to be skewed and/or distorted, the obtained unattended physiological data can be discarded as not representative of the normal physiological state of the patient.

In certain embodiments, the patient monitoring systems 102 further obtain baseline data for patients cared for by the medical institution. Baseline data includes attended physiological data and patient scores, such as an early warning score (EWS) or a modified early warning score (MEWS), and is typically obtained during ward rounds of a clinician. Attended physiological data is obtained with the supervision of a clinician and is indicative of measurements of physiological parameters (or vital signs) of the patients, such as heart rate, respiratory rate, and the like. Attended physiological data is typically obtained wholly or partially from the user input devices 116. However, it is contemplated that the attended physiological data is received by other means. For example, the attended physiological data can be obtained from the sensors 112, provided a clinician ensures the patient does not taint the physiological data. Since attended physiological data is collected with clinician supervision and/or input, attended physiological data can further include manual assessments of physiological parameters of the patient, such as level of consciousness, concern, pain, urine output, and other data pertaining to physiological parameters that cannot be measured by one of the sensors, and/or manual measurements of physiological parameters, such as temperature, respiration rate, and so on.

A patient score assesses the current status of the patient (or, in extreme conditions, the risk of death of a patient) and is obtained through calculation using the attended physiological data and a scoring table of physiological parameters. Suitably, the attended physiological data includes measurements for each physiological parameter of the scoring table. However, attended physiological data including fewer than all of the physiological parameters of the scoring table are contemplated. Scoring tables are completely user configurable, and no assumption regarding the used parameters and scoring thresholds are made. In certain embodiments, the patient monitoring systems 102 facilitate the generation of a patient score. For example, it is contemplated that a process in a processor-based controller 120 of the patient monitoring systems 102 automatically calculates a patient score based on obtained attended physiological data and a scoring table. As another example, it is contemplated that the patient monitoring systems 102 merely provide a clinician with the scoring table and/or the attended physiological data, thereby leaving it to the clinician to calculate the patient score and input it into the relevant patient monitoring system via the user input devices 116.

The scoring table is suitably obtained from a remote component of the IT infrastructure 100, such as the patient information system 104, the patient information display systems 106, or the deterioration detection system 108, via the communications network 110. In such embodiments, a graphical user interface displayed on the remote component can be employed to facilitate selection and/or definition of the scoring table. However, it also is contemplated the scoring table is obtained from one or more memories 118 of the patient monitoring systems 102 and/or selected and/or defined by a clinician via the user input devices 116. As to selection and/or definition, a graphical user interface on the displays 114 of the patient monitoring systems 102 can be employed to facilitate selection and/or definition of the scoring table. Regardless of where the scoring table is obtained from, the scoring table is suitably selected and/or defined individually for each patient based on attributes of the patient, such as illness and/or history.

With reference to FIG. 2, an example of a scoring table for determining a patient score is provided. The first column identifies physiological parameters employed to calculate a patient score, and the first row identifies the score to attribute to measured values of physiological parameters. Each of the cells, other than the cells of the first row and the first column, is associated with the physiological parameter of its row of the cell and the score of the column of the cell. Even more, each of the cells, other than the cells of the first row and the first column, includes a range of measured values for the physiological parameter and score associated with the cell. A score for a measured value of a physiological parameter is determined by matching the value to a cell in the row associated with the physiological parameter and having a range matching the value. The patient score is thus determined by summing the scores of each of the measured values in the attended physiological data, or using the worst case of the individual scores or other rule definitions defined by the scoring schema (e.g., aggregated MEWS and single parameter EWS).

Referring back to FIG. 1, upon obtaining baseline data and/or unattended physiological data, the patient monitoring systems 102 typically relay the baseline data and/or the unattended physiological data to the patient information system 104 and/or other components of the IT infrastructure 100, such as the patient information display systems 106 and/or the deterioration detection system 108, via the communications network 110. However, insofar as the communications network 110 is unavailable, the baseline data and/or the unattended physiological data are suitably buffered in one of the memories 118 of the patient monitoring systems 102 until the communications network 110 is available. The communications network 110 can be unavailable to a patient monitoring system if, for example, the patient monitoring system is outside the range of wireless hot spots of the communications network.

In certain embodiments, the patient monitoring systems 102, in addition to relaying baseline data and/or unattended physiological data, monitor the patients based on the received baseline data and/or unattended physiological data and/or update associated displays to graphically present the baseline data and/or unattended physiological data to clinicians. As to the former, when baseline data and/or unattended physiological data indicates a patient needs medical attention due to, for example, increasing and/or decreasing respiration rate or blood pressure, the patient monitoring systems 102 that received the baseline data and/or unattended physiological data typically generates audio and/or visuals alerts and/or messages notifying clinicians thereof. It is contemplated that message can be provided to the clinicians via the communications network 110.

To carry out the above noted functionality, the patient monitoring systems 102 suitably include the memories 118 and one or more processor-based controllers 120. In certain embodiments, it is contemplated that the patient monitoring systems 102 include patient worn monitors and/or beside monitors. The memories 118 store executable instructions for performing one or more of the above noted functions of the patient monitoring systems 102. Further, in certain embodiments, the memories 118 act as a buffer for the physiological data before it is relayed to the patient information system 104 or some other component of the IT infrastructure 100. This is advantageous when, for example, the patient monitoring systems 102 are not connected to the communications network 110 all the time. The physiological data can be buffered and relayed when a connection to the communications network 110 becomes available. The processor-based controllers 120 execute the executable instructions stored on the memories 118 to carry out the functions associated with the patient monitoring systems 102. Where the patient monitoring systems 102 are operative to relay physiological data over the communications network 110, the patient monitoring systems 102 further include one or more communications units 122 facilitating communication between the processor-based controllers 120 and the communications network 110.

The patient information system 104, such as a central record medical database, receives baseline data and/or unattended physiological data for the patients and stores the data in one of one or more memories 124 thereof. Typically, the data is received from components of the IT infrastructure 100, such as the patient monitoring systems 102 and/or the patient information display systems 106, via the communications network 110. However, it is also contemplated that the data is received via one or more user input devices 126 of the patient information system 104. To facilitate receipt of such data, the patient information system 104 can include a display 128 presenting a user with a graphical user interface. In certain embodiments, the patient information system 104 further displays and/or allows manipulation of the baseline data and/or unattended physiological data in the memories 124 using the user input devices 126 and/or the display 128. Additionally or alternatively, in certain embodiments, the patient information system 104 further allows components of the IT infrastructure 100 to access the data in the memories 124 via the communications network 110.

To carry out the above noted functionality, the patient information system 104 suitably includes the memories 124 and one or more processor-based controllers 130. In certain embodiments, it is contemplated that the memories 124 and the processor-based controllers 130 define one or more computer servers. The memories 124 store executable instructions for performing one or more of the above noted functions of the patient information system 104. Further, as noted above, the memories 124 store baseline data and/or unattended physiological data. The processor-based controllers 130 execute the executable instructions stored on the memories 124 to carry out the functions associated with the patient information system 104. Where the patient information system 104 is operative to receive physiological data from the communications network 110, the patient information system 104 further includes one or more communications units 132 facilitating communication between the processor-based controllers 130 and the communications network 110.

The patient information display systems 106 receive baseline data and/or unattended physiological data for the patients cared for by the medical institution over the communications network 110 from a component of the IT infrastructure 100, such as the patient monitoring systems 102 and/or the patient information system 104. Typically, each of the patient information display systems 106 receives baseline data and/or unattended physiological data for a plurality of patients, but a patient information display system that receives baseline and/or unattended physiological data for a single patient is contemplated. Using the received data, the patient information display systems 106 monitor the patients and/or update associated displays 134 to graphically present the data to clinicians. As to the former, when data indicates a patient needs medical attention due to, for example, increasing and/or decreasing respiration rate or blood pressure, the patient information display systems 106 that received the data typically generates audio and/or visuals alerts and/or messages notifying clinicians.

In certain embodiments, the patient information display systems 106 further allow clinicians to input baseline data via one or more user input devices 136. It is contemplated that graphical user interfaces presented on the displays 134 can be employed to make it easier for the clinicians to enter the data. Upon inputting baseline data, the baseline data is suitably relayed to the patient information system 104 and/or other components of the IT infrastructure 100, such as the deterioration detection system 108, via the communications network 110. Additionally or alternatively, in certain embodiments, the patient information display systems 106 include one or more of nursing stations, bedside monitors, mobile patient information displays, a central monitoring station, a PDA, a tablet computer, a pager, and the like.

To carry out the above noted functionality, the patient information display systems 106 suitably include one or more communications units 138, one or more memories 140, and one or more processor-based controllers 142. The communications units 138 facilitate communication between the processor-based controllers 142 and the communications network 110. The memories 140 store executable instructions for controlling a processor of the processor-based controllers 142 to perform one or more of the above noted functions of the patient information display systems 106. Further, in certain embodiments, the memories 140 act as a buffer for the baseline data before it is relayed to the patient information system 104 or some other component of the IT infrastructure 100. The processor-based controllers 142 execute the executable instructions stored on the memories 140 to carry out the functions associated with the patient information display systems 106.

The deterioration detection system 108 obtains baseline data for the patients from components of the IT infrastructure 100, such as the patient information system 104 and/or the patient monitoring systems 102, and/or one or more user input devices 144 of the deterioration detection system 108, and tracks the most recent baseline data for each of the patients. The baseline data is typically obtained when clinicians make ward rounds and need not be received at predefined intervals. In that regard, the baseline data can be obtained asynchronously. Further, the baseline data suitably represents a complete assessment of the patient for which it relates. In other words, attended physiological data of baseline data suitably includes measurements for each of the physiological parameters employed by the scoring table used to generate the patient score of the baseline data. In certain embodiments, where the age of the most recent baseline data exceeds a predetermined amount, the deterioration detection system 108, for example, generates an audio and/or visual alert and/or sends a message to, for example, a clinician, via the communications network 110 and a pager, PDA, laptop or tablet computer, or the like.

In certain embodiments, upon obtaining baseline data, it is compared against previously obtained baseline data to detect deterioration. It is contemplated that this can be performed through comparison of the patient scores or through comparison of individual physiological parameters, as described below. Additionally or alternatively, in certain embodiments, a plurality of retrospective baseline data is obtained at the same time. For example, the patient monitoring systems 102 buffered the retrospective baseline data until a connection to the communications network 110 was available. In such embodiments, the baseline data can be compared to detect deterioration. Insofar as deterioration is detected, varying actions can be taken based upon the degree of difference between the scores. Actions include one or more of generating an audio and/or visual alert of patient deterioration, logging the deterioration in a database, sending a message and/or page to a clinician via, for example, the communications network 110, and so on.

With reference to FIG. 3, an example timeline 300 illustrating comparison of baseline data for a patient is provided. At time T1, first data 302, including first baseline data or first unattended physiological data, are obtained by a component of the IT infrastructure 100, where the patient's condition is designated by a first “X” 304. At time T2, second data 306, including second baseline data or second unattended physiological data, are obtained by a component of the IT infrastructure 100, where the patient's condition is designated by a second “X” 308. Initially, at times T1 and T2, the first data 302 and the second data 306 are only available at their respective component. These components either upload the data to the deterioration detection system 108 immediately or timely delay the upload until a connection with the deterioration detection system 108 is available. Because delay is possible, the first data 302 could be received after the second data 306. Upon receiving the first data 302 and the second data 306, the deterioration detection system 108 compares the data. As shown by the change of the patient's condition from time T1 to time T2, deterioration has occurred.

Referring back to FIG. 1, typically during the interim between baseline data, the deterioration detection system 108 obtains unattended physiological data. The unattended physiological data is suitably obtained intermittently, such as periodically, in any random time sequence and/or continuously from components of the IT infrastructure 100, such as the patient information system 104 and/or the patient monitoring systems 102. However, it is to be appreciated that the unattended physiological data can be obtained asynchronously. In contrast with baseline data, unattended physiological data typically does not represent a complete assessment of the patient for which it relates. Rather, it typically includes a subset of the physiological parameters employed by the scoring table for the patient. In certain embodiments, the deterioration detection system 108 sends network events to the patient monitoring systems 102 requesting the unattended physiological data. For example, the deterioration detection system 108 can request the physiological data if a predetermined amount of time has elapsed since last receiving unattended physiological data. Although described as a separate unit, it is to be appreciated that the deterioration detection system 108 can be incorporated into the patient monitoring systems 102 or other network components.

Although the unattended physiological data is typically obtained intermittently and/or continuously, in certain embodiments, the unattended physiological data is retrospective and/or obtained asynchronously. For example, the patient monitoring systems 102 can buffer the physiological data until a connection to the communications network 110 is available. Where the unattended physiological data is retrospective, typically only the most recent measurements are considered and/or a trending algorithm (e.g., average, maximum, etc.) is applied to the physiological data. However, it is also contemplated that, retrospective data can also be employed to detect patient deterioration in the past in the same manner discussed below. In such embodiments, each retrospective unattended physiological data received is compared against the most recent retrospective baseline data temporally preceding it.

With reference to FIG. 4, an example timeline 400 illustrating a patient's condition via a trend line 402 and the receipt of baseline data 404 (designated by a long bar) and unattended physiological data 406 (designated by a short bar) is provided. The unattended physiological data 406 is received intermittently during the interim between the baseline data 404 upon the happening of, for example, timer events, manual trigger events, etc. Also illustrated, a patient monitoring system providing the unattended physiological data 406 loses its connection to the communications network 110 at time T1. Thereafter, at time T2, the connection is restored and the unattended physiological data 408 generated between time T1 and time T2 is provided to the deterioration detection system 108. While this example assumes a single patient monitoring system, it is to be appreciated that multiple patient monitoring systems can provide physiological data for a patient.

Referring back to FIG. 1, upon obtaining the unattended physiological data, the deterioration detection system 108 compares it to most recent baseline data using the scoring table. As noted above, the baseline data includes measured values for physiological parameters and each measured value is scored using the scoring table. Insofar as no baseline data is available, a virtual baseline including the least severe scores of the physiological parameters of the scoring table is assumed. Each measured value of a physiological parameter in the unattended physiological data is similarly scored and compared with the score of the corresponding measured value in the latest baseline data (or virtual baseline). The difference between the scores is compared against one or more thresholds to determine if, and what, action should be taken. Varying actions can be taken based upon the degree of difference between the scores. Actions include one or more of generating an audio and/or visual alert of patient deterioration, logging the deterioration in a database, sending a message and/or page to a clinician via, for example, the communications network 110, and so on.

In certain embodiments, before action is taken, the deterioration detection system 108 seeks reassurance that the unattended physiological values are not skewed and/or distorted by movement artifacts and/or from not knowing under which condition the measurements were done. When the unattended physiological data is obtained continuously, measurements can be captured at a predetermined interval for a predetermined number of times. When the unattended physiological data is not obtained continuously, the deterioration detection system can request the patient monitoring systems 102 to repeat measurements at a predetermined interval or timely repetition patterns and/or sequences for a predetermined number of times. To request the patient monitoring systems 102 to repeat measurements, the deterioration detection system 108 can send a network event to the patient monitoring systems 102 requesting unattended physiological data as to one of the deteriorated measurement, a subset of all measurements, all measurements, or the like. Upon capturing or obtaining the measurements, they can be employed to determine whether a patient was in an intermittent state or if the measurements were representative for the patient.

The amount of time and the number of repetitions is adapted to the individual environment, including the kinds of measurement, as well as level of care and general patient class. Further, the amount of time and the number of repetitions is suitably obtained from a clinician. In that regard, the user input devices 144 of the deterioration detection system 108 can be employed to capture these parameters from the clinician. In certain embodiments, a graphical user interface of the deterioration detection system 108 can be presented to a clinician via a display 146 to facilitate such a task. Alternatively, other components of the IT infrastructure 100 can be employed to obtained the amount of time and the number repetitions from the clinician. While the amount of time and the number of repetitions are suitably obtained from the clinician, automated approaches to defining these parameters are contemplated. For example, algorithms based on one or more of the scoring table; actual score or sub-scores; severity of deterioration; additional available (e.g., automatically captured) status information of the patient, such as current patient activity, activity trend, and posture; and so on, can be employed to automatically define the parameters.

With reference to FIG. 5, a table of events illustrative of deterioration detection for a patient is provided. The patient score was evaluated using the scoring table of FIG. 2, whereby attention is also directed thereto. At 5:25 AM, baseline data is received, which indicates the patient is doing well and has a patient score of zero. Twelve hours later, at 5:25 PM, baseline data is received and again the patient is doing well. In the meantime at, for example, automatically and/or manually triggered intervals, unattended physiological data are received and are compared to baseline data using the scoring table, but deterioration is not detected. At 7:25 PM, unattended physiological data for blood pressure (BP) and heart rate (HR) are obtained. Scoring the blood pressure using the scoring table, the blood pressure has a score equal to that of the most recent baseline data (i.e., the baseline data received at 5:25 PM). Therefore, there is no deterioration with blood pressure. However, scoring the heart rate using the scoring table, the heart rate has a score of one, as compared with the score of zero of the most recent baseline data. Therefore, deterioration has been detected. At this point, the deterioration detection system 108 can seek reassurance or take the appropriate action, such as notifying a clinician. Assuming notice was given, the clinician typically checks on the patient and updates the baseline data, as shown at 8:10 PM. Thereafter, unattended physiological data is obtained at 11:30 PM. Compared to the most recent baseline data (i.e., the baseline data at 8:10 PM), both blood pressure and heart rate have deteriorated. As above, the deterioration detection system 108 can seek reassurance or take the appropriate action, such as notifying a clinician. Again, assuming notice was given, updated baseline data is typically obtained, as shown at 11:45 PM. However, this time the patient is unresponsive.

To carry out the above noted functionality, the deterioration detection system 108 suitably includes a communication unit 148, a memory 150, and a processor-based controller 152. The communications unit 148 facilitates communication between the processor-based controller 152 and the communications network 110. The memory 150 stores executable instructions for controlling the processor of the processor-based controller 152 to perform one or more of the above noted functions of the patient deterioration system 108. The processor-based controller 152 executes the executable instructions stored on the memory 150 to carry out the functions associated with the patient deterioration system 108. In certain embodiments, the patient deterioration system 108 further includes the user input devices 144 and/or the display 146 allowing a clinician to manually enter baseline data and/or other parameters employed by the deterioration detection system 108.

While the deterioration detection system 108 is shown as a separate component of the IT infrastructure 100, it is to be appreciated that it can be integrated with other components of the IT infrastructure 100. For example, the deterioration detection system 108 can be integrated with the patient information system 104. As another example, the deterioration detection system 108 can be integrated with one or more of the patient monitoring systems 102.

With reference to FIG. 6, a flow chart of a method 600 for detecting patient deterioration is provided. The method 600 is suitably performed for multiple patients simultaneously. In that regard, a scoring table is identified 602 for each of the patients based on patient history and illness. Suitably, identification includes selection from a predefined collection of scoring tables or definition of a new scoring table. Further, a clinician suitably performs selection and/or definition.

After identification 602 of the scoring table for a patient, baseline data is generated 604 intermittently for the patient by a clinician. Baseline data includes attended physiological data and a patient score. The attended physiological data includes data indicative of manually or automatically collected measurements of physiological parameters of the patient and, optionally, data indicative of manual assessments of physiological parameters of the patient. The patient score is generated automatically or manually from the scoring table using the attended physiological data.

Unattended physiological data is also collected 606 for a patient. Unattended physiological data includes data indicative of measurements of at least one of the physiological parameters of the scoring table for the patient. Typically, however, unattended physiological data includes a subset of the physiological parameters. Unattended physiological data is collected automatically by, for example, a patient worn device or bed side device. Further, unattended physiological data is typically collected continuously, on-demand, or upon the occurrence of an event, such as a timer event. Where the unattended physiological data is continuously collected, the unattended physiological data can be broken into discrete blocks based on time and trending algorithms can be applied to the discrete blocks.

When new unattended physiological data is collected 606 for a patient, the unattended physiological data is compared 608 against the most recent baseline data for the patient using the scoring table to detect deterioration. Insofar as no deterioration is detected, the method 600 is suspended until further unattended physiological data is collected. Insofar as deterioration is detected, however, the method 600 suitably seeks re-assurance 610 of the unattended physiological data.

Re-assurance 610 checks that the measurements of a patient are really deteriorated and that the patient is in a baseline physiological state. If the patient is not in a baseline physiological state, the measurements may be tainted. In certain embodiments, re-assurance 610 includes triggering additional measurements of at least one of the physiological parameters of the unattended physiological parameters. Suitably, the at least one includes the deteriorated physiological parameters. Additionally or alternatively, in certain embodiments, re-assurance 610 includes capturing measurements at a predetermined interval for a predetermined number of times from a continuous stream of unattended physiological data.

If a patient's condition has deteriorated and, if appropriate, the deterioration has been reassured, a clinician is notified 612 of the deterioration. Suitably, such notification prompts the clinician to take further action 614, such as generating additional baseline data.

With reference to FIG. 7, a block diagram of a method 700 suitably performed by the deterioration detection system 108 is provided. A scoring table is individually selected for each patient based on illness and history. Attended physiological data for the patient is received 702. The attended physiological data includes measurements, automatic, manual or otherwise, of physiological parameters of the patient and/or manual assessments of physiological parameters of the patient. Further, a patient score for the patient is obtained 704 from the attended physiological data and the scoring table. Typically, the patient score is calculated from the attended physiological data and the scoring table by a clinician, but automated approaches are contemplated. Physiological data, including at least one of unattended physiological data and attended physiological data, is received 706 for the patient. Typically, the physiological data is received periodically or continuously. The physiological data includes measurements of one or more of the physiological parameters of the patient.

In certain embodiments, actions are taken to verify that the measurements of the physiological data are representative for the patient. Typically, this is only when the physiological data includes unattended physiological data. For example, verification of the measurements of the physiological data is received from a clinician. As another example, in response to measurements which differ by more than a threshold from earlier measurements, the deterioration detection system 108 controls or otherwise causes one of the patient monitoring systems 102 to retake the measurements in question. As yet another example, additional data, such as data pertaining to motion, patient activity, body posture, and so on, is received from a component of the IT infrastructure 100, such as one of the patient monitoring systems 102. Thereafter, a determination as to whether the physiological data accurately reflects the physiological state of the patient is made based on the additional data. The physiological data is discarded in response to the physiological data not accurately reflecting a baseline physiological state of the patient and taken again.

Additionally or alternatively, in certain embodiments, the physiological data is continuously received. As above, this is typically only when the physiological data includes unattended physiological data. In some of these embodiments, the physiological data is divided 708 into discrete blocks of physiological data based on time. Thereafter, a trending algorithm is applied 710 to each of the discrete blocks, so each of the blocks is associated with a single measurement for each of one or more physiological parameters. Trending algorithms include, for example, average, median, peak-finding, etc.

Upon receiving 706 the physiological data, the measurements of the physiological data are compared 712 to corresponding measurements in most recent attended physiological data using the scoring table to determine any change in the patient score. In certain embodiments, in response to a physiological parameter of the physiological data deteriorating as compared to a corresponding physiological parameter in the most recent attended physiological data, deterioration is verified 714. For example, one of the patient monitoring systems 102 is controlled (via, for example, a network event) to take measurements of at least one of the physiological parameters a predetermined number of times in one or more predetermined intervals or timely repetition patterns and/or sequences. These additional measurements are then used to determine whether the detected deterioration is representative of the patient. A clinician is notified 716 of patient deterioration in response to a physiological parameter of the physiological data deteriorating as compared to a corresponding physiological parameter in the most recent attended physiological data.

With reference to FIG. 8, a block diagram of a method 800 for verifying deteriorated unattended physiological data is provided. Deteriorated unattended physiological data is received 802 for a patient, including measurements of one or more physiological parameters of the patient. In response to intermittently receiving unattended physiological data, a patient monitoring system 102 is controlled 804 to take additional measurements of at least one of the physiological parameters a predetermined number of times in one or more predetermined intervals or timely repetition patterns and/or sequences. Supplemental unattended physiological data is received 806 for a patient, including the additional measurements. In response to continuously receiving unattended physiological data, measurements of at least one of the physiological parameters are captured 808 a predetermined number of times in one or more predetermined intervals or timely repetition patterns and/or sequences. The measurements of the unattended physiological data are compared 810 to corresponding measurements of the supplemental unattended physiological data or the captured measurements.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor-based controller includes one or more of a microprocessor, a microcontroller, a graphic processing unit (GPU), an application-specific integrated circuit (ASIC), a field-programmable gate array (FPGA), and the like; a user input device includes one or more of a mouse, a keyboard, a touch screen display, one or more buttons, one or more switches, one or more toggles, and the like; and a display includes one or more of a LCD display, an LED display, a plasma display, a projection display, a touch screen display, and the like.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A deterioration detection system for detecting deterioration of a patient of a medical institution, said system comprising:
one or more processors programmed to:
receive attended physiological data for a patient, the attended physiological data including measurements of physiological parameters of the patient;
obtain a patient score for the patient from the attended physiological data and a scoring table;
receive physiological data, including at least one of unattended physiological data and attended physiological data, for the patient, the physiological data including measurements of one or more of the physiological parameters of the patient;
compare the measurements of the physiological data to corresponding measurements in most recent attended physiological data using the scoring table to determine any change in the patient score; and,
notify a clinician of patient deterioration in response to one or more physiological parameters of the physiological data deteriorating as compared to corresponding physiological parameters in the most recent attended physiological data

2. The system according to claim 1, wherein the comparing includes:
determining a score for each of at least one of the measurements of the physiological data using the scoring table;
determining a score for each of at least one of the measurements of the most recent attended physiological data using the scoring table; and,
comparing the scores of the physiological data measurements and the most recent attended physiological data corresponding measurements.

3. The system according to Claim 1, wherein the one or more processors are further programmed to:
verify deterioration of the patient in response to a physiological parameter of the physiological data deteriorating as compared to a corresponding physiological parameter in the most recent attended physiological data.

4. The system according to claim 3, wherein the verifying includes:
controlling a patient monitoring system to take measurements of at least one of the physiological parameters a predetermined number of times in one or more predetermined intervals or timely repetition patterns and/or sequences.

5. The system according to claim 1, wherein the physiological data is continuously received, wherein the processors are further programmed to:
divide the physiological data into discrete blocks of physiological data based on time; and,
apply a trending algorithm to each of the discrete blocks.

6. An information technology (IT) infrastructure of a medical institution, said IT infrastructure comprising:
the deterioration detection system according Claim 1;
one or more patient monitoring systems which monitor a patient and generate at least the physiological data; and,
a communications network which facilitates the exchange of at least one of the physiological data, the attended physiological data, and the patient score between the deterioration detection system and the patient monitoring systems.

7. A method for detecting deterioration of a patient of a medical institution, said method comprising:
receiving attended physiological data for the patient, the attended physiological data including measurements of physiological parameters of the patient;
obtaining a patient score for the patient from the attended physiological data and a scoring table;
receiving physiological data, including at least one of unattended physiological data and attended physiological data, for the patient, the physiological data including measurements of one or more of the physiological parameters of the patient;
comparing the measurements of the physiological data to corresponding measurements in most recent attended physiological data using the scoring table to determine any change in the patient score; and,
notifying a clinician of patient deterioration in response to a physiological parameter of the physiological data deteriorating as compared to a corresponding physiological parameter in the most recent attended physiological data.

8. The method according to claim 7, further including:
selecting the scoring table based on illness and history of the patient.

9. The method according to Claim 7, further including at least one of:
comparing the most recent attended physiological data with previously obtained attended physiological data to detect patient deterioration;
comparing most recent physiological data with past physiological data to detect patient deterioration, physiological data including at least one of attended physiological data and unattended physiological data; and,
comparing past physiological data with other past physiological data to detect backdated patient deterioration in the past, physiological data including at least one of attended physiological data and unattended physiological data.

10. The method according to claim 7, wherein the comparison includes:
determining a score for each of at least one of the measurements of the physiological data using the scoring table;
determining a score for each of at least one of the measurement of the most recent attended physiological data using the scoring table; and,
comparing the scores of the physiological data measurements and the most recent attended physiological data corresponding measurements.

11. The method according to claim 7, further comprising:
verifying deterioration of the patient in response to a physiological parameter of the physiological data deteriorating as compared to a corresponding physiological parameter in the most recent attended physiological data,

12. The method according to claim 11, wherein the verifying includes at least one of:
controlling a patient monitoring system to take measurements of at least one of the physiological parameters a predetermined number of times in one or more predetermined intervals or timely repetition patterns and/or sequences; and,
receiving verification of the measurements of the physiological data from a clinician.

13. The method according to claim 7, wherein at least one of the most recent attended physiological data the patient score, and the physiological data is received over a communications network.

14. The method according claim 7, further comprising:
receiving additional data, including at least one of motion data, activity data, and posture data, indicative of whether the patient is in a normal physiological state;
determining whether the physiological data accurately reflects the physiological state of the patient based on the additional data; and,
discarding the physiological data in response to the physiological data not accurately reflecting a baseline physiological state of the patient.

15. The method according to claim 7, wherein the physiological data is continuously received, said method further comprising:
dividing the physiological data into discrete blocks of physiological data based on time and,
applying a trending algorithm to each of the discrete blocks.

16. One or more processors programmed to perform the method according to claim 7.

17. A computer medium carrying software which controls one or more processors to perform the method according to claim 7.

18. A method for verifying deteriorated unattended physiological data for a patient, said method comprising:
receiving the deteriorated unattended physiological data, including measurements of one or more physiological parameters of the patient;
in response to intermittently receiving unattended physiological data:
controlling a patient monitoring system to take additional measurements of at least one of the physiological parameters a predetermined number of times in one or more predetermined intervals; and,
receiving supplemental unattended physiological data for the patient, including the additional measurements;
in response to intermittently and/or continuously receiving unattended physiological data, capturing measurements of at least one of the physiological parameters a predetermined number of times in one or more predetermined intervals or timely repetition patterns and/or sequences; and,
comparing the measurements of the deteriorated unattended physiological data to corresponding measurements of the supplemental unattended physiological data or the captured measurements.

19. One or more processors programmed to perform the method according to claim 18.

20. A deterioration detection system for detecting deterioration of a patient of a medical institution, said system comprising:
one or more processors programmed to:
receive attended physiological data for a patient, the attended physiological data including measurements of physiological parameters of the patient;
obtain a patient score from the attended physiological data and a scoring table;
receive unattended physiological data for the patient, including measurements of one or more of the physiological parameters of the patient;
compare the measurements of the unattended physiological data to corresponding measurements in most recent attended physiological data using the scoring table to determine any change in patient score; and
verify the unattended to physiological according to the method of claim 18 in response to a physiological parameter of the unattended physiological data deteriorating as compared to a corresponding physiological parameter in the most recent attended physiological data.
